(19) United States
(12) Patent Application Publication
(10) Pub. No.: US 2020/0032249 A1
(43) Pub. Date: Jan. 30, 2020

(54) LABELING, ISOLATION, & ANALYSIS OF RNA FROM RARE CELL POPULATIONS

(71) Applicant: MEMORIAL SLOAN KETTERING CANCER CENTER, New York, NY (US)

(72) Inventors: Harihar BASNET, New York, NY (US); Joan Massagué, New York, NY (US)

(21) Appl. No.: 16/493,716

(22) PCT Filed: Mar. 13, 2018
(86) PCT No.: PCT/US2018/022092

§ 371 Date: Sep. 12, 2019

Related U.S. Application Data
(60) Provisional application No. 62/471,264, filed on Mar. 14, 2017.

(51) Int. Cl. C12N 15/10; C12N 15/85

(52) U.S. Cl. CPC C12N 15/1065; C12Y 204/02009; C12N 15/85

(57) ABSTRACT

The present invention provides new and improved methods for performing in situ transcriptomics from rare “cells of interest” present in complex multicellular environments. These methods involve expressing a recombinant cytosine deaminase enzyme in the cells of interest, and also supplying an exogenous non-naturally occurring halogenated cytosine substrate for the enzyme—which results in the generation halogenated uridine which is incorporated into RNA, thereby “tagging” RNA in the cells of interest. The invention also provides several variations of such methods that significantly improve the sensitivity and specificity of the RNA labeling. Furthermore, the invention also provides simple and efficient methods for purifying the tagged RNA. The purified tagged RNA can be used to analyze the transcriptomes of the cells of interest by RNA-sequencing and other methods.

### CROSS-REFERENCE TO RELATED APPLICATIONS

This application claims the benefit of priority of U.S. Provisional Patent Application No. 62/471,264 filed on Mar. 14, 2017, the content of which is hereby incorporated by reference in its entirety.

### STATEMENT OF GOVERNMENT SUPPORT

This invention was made with government support under grant number W81XWH-12-1-0074 awarded by the U.S. Army Medical Research and Materiel Command. The government has certain rights in the invention.

### INCORPORATION BY REFERENCE

For the purpose of only those jurisdictions that permit incorporation by reference, all of the references cited in this disclosure are hereby incorporated by reference in their entireties (numbers in parentheses or in superscript following text in this patent disclosure refer to the numbered references provided in the “Reference List” section of this patent specification). In addition, any manufacturers' instructions or catalogues for any products cited or mentioned herein are incorporated by reference. Documents incorporated by reference into this text, or any teachings therein, can be used in the practice of the present invention.

### BACKGROUND

Tissues are comprised of different cell types whose interactions elicit distinct gene expression patterns that regulate tissue formation, regeneration, homeostasis and repair. Analysis of these gene expression patterns requires methods that can capture as closely as possible the transcriptomes of cells of interest in their tissue microenvironment. The tissue microenvironment plays a crucial role in determining gene expression in cells in vivo, yet tools that can accurately and sensitively capture the transcriptomics of rare cells of interest in the context of an intact tissue microenvironment are lacking. While currently available techniques such as TRAP-seq and TU-tagging can capture in situ transcriptomics, these methods are limited in their ability to study rare cell populations due to their inherent noise (1-3). The application of TU-tagging is limited to cell populations that constitute at least 5% of the total tissue (1), while TRAP-seq has slightly higher sensitivity and can be used for cell populations that constitute at least 1% of the total population (2). Furthermore, current technologies designed to study in situ transcriptomics are limited by the requirement to perform multiple steps after tissue dissociation (3-5), and the requirement for the use of sophisticated tools (6), making it challenging to transcriptionally profile rare cell populations rapidly isolated from their native microenvironment. Many crucial questions of tremendous biological and clinical significance involve cell populations representing far fewer than 1% of the total cells in a tissue, such as adult stem cells, subtypes of neuronal cells and immune cells, dormant cancer cells, and others. As such, there is a need in the art for new and improved methods for studying in situ transcriptomics in rare cell populations. The present invention addresses this need.

### SUMMARY OF THE INVENTION

The present invention provides new and improved methods for labeling RNA and performing in situ transcriptomics from cell populations, including rare cell populations, present within complex multicellular environments.

The enzyme cytosine deaminase or “CD” is naturally expressed in prokaryotes and fungi, but not in mammalian cells. The CD enzyme converts cytosine to uracil, which can then be converted by further enzymatic action to uridine and uridine triphosphate—which can then be incorporated into RNA. The present invention exploits the activity of the CD enzyme to selectively label/tag RNA in cells of interest present in mixed cell populations. In particular, the methods of the present invention involve expressing a recombinant CD enzyme in mammalian cells of interest, and also supplying an exogenous non-naturally occurring labeled/tagged substrate for the CD enzyme—specifically a halogenated cytosine, such as, for example, 5-fluoro-cytosine. Using this system, non-native metabolites of the halogenated cytosine substrate (e.g. halogenated uracil, halogenated uridine, and halogenated uridine triphosphate) are generated and incorporated into RNA in the cells of interest—thereby specifically labeling/tagging newly synthesized RNAs with halogenated uridine derivatives. Because mammalian cells cannot convert cytosine (or halogenated cytosine derivatives) to uracil without expression of the recombinant CD enzyme, this CD-based RNA tagging system can be used to selectively tag RNA in the cells of interest in a highly controlled manner.

While others have suggested employing CD to label RNA with reactive moieties (see WO 2016/154040), to the best of Applicant's knowledge, such an approach has never been successfully performed. In contrast, and as shown in the Examples section of the present application, Applicants have now successfully demonstrated that CD-based labeling of RNA with halogen atoms can be successfully performed. Importantly, such methods were found to be effective even though halogenated uracil derivatives are widely known to be toxic. For example, 5-fluoro uracil is highly cytotoxic, and is, in fact, used extensively as a chemotherapeutic agent for the treatment of tumors—based on its ability to induce cell death by inhibiting thymidylate synthase and by causing DNA and RNA damage (12-14). Nonetheless, the inventors have found that halogenated uracil derivatives can, surprisingly, be used to efficiently label RNA in the present methods. Indeed, the inventors have also demonstrated that efficient RNA labeling can even be achieved when the timing and dosing of the halogenated cytosine exposure is reduced to a level that causes minimal-to-no cytotoxicity. Furthermore, the use of halogenated cytosine (such as fluoro-cytosine) as the substrate in these CD-based RNA tagging methods appears to provide several advantages over other potential tagging and/or labeling systems. For example, because the tag is very small in size (a single halogen atom), the tagged cytosine remains a suitable substrate for the cytosine deaminase enzyme—and can be effectively converted to halogenated uracil by the CD enzyme. This is in contrast to labeling/tagging of cytosine with larger tags, (such as multi-atomic reactive moieties), which may compromise the ability of the cytosine deaminase enzyme to convert the labeled/tagged cytosine to uracil and thus severely reduce the efficiency of, or even prevent, RNA labelling. (Indeed, preliminary testing data suggests that the efficiency of conversion of cytosine to uracil by CD is inversely correlated with the size of any tag present on the cytosine molecule).

In some embodiments the CD-based RNA tagging systems of the present invention summarized above also involve expressing recombinant uracil phosphoribosyl transferase (UPRT) in the cells of interest. This limits the diffusion of halogenated uracil to neighboring cells—thus increasing the signal-to-noise ratio of the present methods.

In some embodiments the CD-based RNA tagging systems of the present invention summarized above also involve supplying exogenous thymine to the cells of interest. The thymine acts as a competitive inhibitor of halogenated uracil/halogenated uridine export from the CD-expressing cells—thus further increasing the signal-to-noise ratio of the present methods.

In some embodiments the tagged RNAs generated in the cells of interest using the CD-based RNA tagging systems of the present invention can be detected and/or purified very efficiently using antibodies that bind to tagged uridine-containing RNAs specifically, but that do not bind to (or have significantly lower levels of binding to) tagged cytidine containing RNAs. Such antibodies include, but are not limited to, anti-bromodeoxyuridine (“BrdU”) antibodies, which bind specifically to halogenated-uridine containing nucleic acid molecules. Anti-BrdU antibodies are well known in the art and are widely available. The use of such antibodies to detect and/or purify RNA from the cells of interest provides several important advantages. For example, the use of such antibodies for RNA purification reduces or eliminates contamination by RNA that contains the halogenated cytosine substrate or its cytidine derivatives (e.g. cytidine triphosphate). This is in contrast to detection and/or purification methods that are based purely on the existence of the tag itself (whether that is a reactive moiety or any other type of tag)—irrespective of whether the tag is present on a cytidine derivative or on a uridine derivative. This is important because derivatives of the tagged cytosine substrates used in the present methods can be incorporated into RNA in a CD-independent manner—such that cells other than the cells of interest contain tagged RNA. Avoiding contamination by RNA that contains tagged cytidine is particularly advantageous where the cells of interest are very rare. Furthermore, the use of such antibodies eliminates the need to perform additional steps and/or utilize additional chemistry before the tagged RNA can be detected and/or purified. This is in contrast to RNA detection and purification systems that involve, for example, tagging RNA with a reactive moiety and then performing chemical reactions to render the RNA detectable.

The purified tagged RNAs obtained using the methods described herein (which represent the transcriptomes of the cells of interest), can be analyzed using any desired technique used for RNA analysis, including, but not limited to, microarray analysis, RT-PCR and/or RNA-sequencing (“RNA-seq”).

Using the new and improved methods of the present invention it is now possible to obtain and define the transcriptomes of cells of interest representing as few as 0.003% of the total cell population in an organ or tissue. As such, the methods of the present invention can be used to study the transcriptomes of rare cell populations including, but not limited to, stem cells, micro-metastatic cells, post-treatment residual cancer cells, sub-populations of neurons, and specific types of immune cells, and can also be used to study such cell types in a variety of different situations, including in response to diverse stimuli and/or in multiple different physiological and pathological contexts. Furthermore, because the methods of the invention only measure newly synthesized RNAs, the methods can be used to identify rapid changes in gene expression in cells of interest in response to a variety of external factors such as ligands, hormones, and drugs.

In some embodiments, the present invention provides a method of producing tagged RNA from mammalian cells of interest present in a tissue or tissue culture that contains multiple cell types, the method comprising contacting a tissue or a tissue culture that contains multiple cell types with an effective amount of a halogenated cytosine, wherein mammalian cells of interest in the tissue or tissue culture have been engineered to express a recombinant cytosine deaminase enzyme, thereby generating halogenated uridine-tagged RNA in the cells of interest.

In some such embodiments such methods may further comprise subsequently separating halogenated uridine-tagged RNA from other components of the tissue, tissue culture, and/or cells of interest. Similarly, in some such embodiments such methods may further comprise subsequently contacting a cell lysate or RNA sample derived from the tissue or tissue culture with an antibody that binds specifically to halogenated uridine-tagged RNA. Similarly, in some such embodiments such methods may further comprise subsequently separating halogenated uridine-tagged RNA from other components in the cell lysate or RNA sample based on its binding to the antibody, thereby obtaining halogenated uridine-tagged RNA from the cells of interest present in the tissue or tissue culture.

In some embodiments, the present invention provides a method of obtaining tagged RNA from mammalian cells of interest present in a tissue or tissue culture that contains multiple cell types, the method comprising: (a) contacting a tissue or a tissue culture that contains multiple cell types with an effective amount of a halogenated cytosine, wherein mammalian cells of interest in the tissue or tissue culture have been engineered to express a recombinant cytosine deaminase enzyme; (b) contacting a cell lysate or RNA sample derived from the tissue or tissue culture with an antibody that binds specifically to halogenated uridine-tagged RNA; and (c) separating halogenated uridine-tagged RNA from other components in the cell lysate or RNA sample based on its binding to the antibody; thereby obtaining tagged RNA from the cells of interest present in the tissue or tissue culture.

Each of the embodiments described above, or elsewhere herein, may, optionally, further comprise contacting the tissue or tissue culture with an effective amount of exogenous thymine.

Each of the embodiments described above, or elsewhere herein, may, optionally, be performed using cells of interest that also express a recombinant uracil phosphoribosyltransferase (UPRT) enzyme.

In some embodiments, the present invention provides a method of producing tagged RNA from mammalian cells of interest present in a tissue or tissue culture that contains multiple cell types, the method comprising: (a) contacting a tissue or a tissue culture that contains multiple cell types with an effective amount of a halogenated cytosine, wherein mammalian cells of interest in the tissue or tissue culture have been engineered to express a recombinant cytosine deaminase enzyme, and (b) contacting the tissue or a tissue culture with an effective amount of exogenous thymine, thereby producing tagged RNA from the cells of interest present in the tissue or tissue culture.

In some embodiments, the present invention provides a method of producing tagged RNA from mammalian cells of interest present in a tissue or tissue culture that contains multiple cell types, the method comprising: (a) contacting a tissue or a tissue culture that contains multiple cell types with an effective amount of a halogenated cytosine, wherein mammalian cells of interest in the tissue or tissue culture have been engineered to express both (i) a recombinant cytosine deaminase enzyme, and (ii) a recombinant uracil phosphoribosyltransferase (UPRT) enzyme, and (b) contacting the tissue or a tissue culture with an effective amount of an exogenous thymine, thereby producing tagged RNA from the cells of interest present in the tissue or tissue culture.

Each of the embodiments described above, or elsewhere herein, may, optionally, further comprise performing RNA sequencing of the tagged RNA, and/or reverse transcribing the tagged RNA to produce cDNA, and/or performing RT-PCR with the tagged RNA, and/or amplifying the tagged RNA, or cDNA derived therefrom, and/or performing microarray analysis of the tagged RNA.

In each of the embodiments described above, or elsewhere herein, the halogenated cytosine may be selected from the group consisting of fluoro-cytosine, chloro-cytosine, bromo-cytosine, and iodo-cytosine. For example, in some embodiments the halogenated cytosine is 5-fluoro-cytosine and the halogenated uridine-tagged RNA is 5-fluoro-uridine-tagged RNA.

In each of the embodiments described above, or elsewhere herein, the methods may, optionally, also comprise contacting a cell lysate or RNA sample derived from the tissue or tissue culture with an anti-BrdU antibody.

In each of the embodiments described above, or elsewhere herein, the methods may, optionally, also comprise performing immuno-affinity chromatography or immunoprecipitation to separate halogenated uridine-tagged RNA from other components in the cell lysate or RNA sample. For example, in some embodiments two successive rounds of immuno-affinity chromatography or immunoprecipitation may be performed to separate the halogenated uridine-tagged RNA from other components in the cell lysate or RNA sample.

In each of the embodiments described above, or elsewhere herein, the cells of interest used in the methods of the invention may, optionally, comprise a recombinant nucleic acid molecule that comprises a nucleotide sequence encoding a cytosine deaminase enzyme operatively linked to a promoter. Similarly, in each of the embodiments described above, or elsewhere herein, the cells of interest used in the methods of the invention may, optionally, comprise a recombinant nucleic acid molecule that comprises a nucleotide sequence encoding a UPRT enzyme operatively linked to a promoter. In some embodiments the cells of interest may, optionally, comprise both (a) a recombinant nucleotide sequence encoding a cytosine deaminase enzyme, and (b) a recombinant nucleotide sequence encoding a UPRT enzyme. In some such embodiments the nucleotide sequence encoding the cytosine deaminase enzyme and the nucleotide sequence encoding the UPRT enzyme may, optionally, be present on the same nucleic acid molecule, for example on a nucleic acid molecule comprising an internal ribosome entry site (IRES) sequence or viral 2A peptide encoding sequence located between the nucleotide sequence encoding the cytosine deaminase enzyme and the nucleotide sequence encoding the UPRT enzyme. Alternatively, in other embodiments, the nucleotide sequence encoding the cytosine deaminase enzyme and the nucleotide sequence encoding the UPRT enzyme may each be present on a separate nucleic acid molecule. In each of such embodiments the nucleotide sequence encoding the cytosine deaminase enzyme, and/or the nucleotide sequence encoding the UPRT enzyme may, optionally, comprise an inducible promoter, and/or a tissue-specific promoter.

In each of the embodiments described above, or elsewhere herein, the cells of interest may be in vitro or in vivo.

In embodiments where the cells of interest are in vivo the cells may be contacted with the halogenated cytosine in vivo. In some embodiments the cells of interest may have been injected into a living animal or may be derived from cells that have been injected into a living animal. In some embodiments the cells of interest may be present in a genetically engineered animal that has been engineered to express the recombinant cytosine deaminase enzyme, and/or to express both the recombinant cytosine deaminase enzyme and UPRT.

In embodiments where the cells of interest are in vitro the cells may be contacted with the halogenated cytosine in vitro. For example, a tissue or tissue culture comprising the cells of interest may be contacted in vitro with the halogenated cytosine at a concentration of up to about 50 micro molar, or of up to about 125 micro molar, or of up to about 250 micro molar, or of up to about 500 micro molar.

In embodiments where the cells of interest are in vivo the cells may be contacted with the halogenated cytosine in vivo. For example, in some embodiments a dose of up to about 50 mg/kg, or up to about 125 mg/kg, or up to about 250 mg/kg, or up to about 500 mg/kg, of the halogenated cytosine may be administered to an animal comprising the cells of interest.

In some embodiments, the cells of interest, or the tissue or tissue culture, may be contacted with the halogenated cytosine in vitro or in vivo for a period of at least about 2 hours. In some embodiments, the cells of interest, or the tissue or tissue culture, may be contacted with the halogenated cytosine in vitro or in vivo for or for a period of up to about 48 hours, or for a period of up to about 24 hours, or for a period of up to about 12 hours, or for a period of up to about 8 hours, or for a period of up to about 6 hours, or for a period of up to about 4 hours. In some embodiments, the cells of interest, or the tissue or tissue culture, may be contacted with the halogenated cytosine in vitro or in vivo for a period of from about 2 hours to about 24 hours, or for a period of from about 2 hours to about 12 hours, or for a period of from about 2 hours to about 8 hours, or for a period of from about 2 hours to about 6 hours, or for a period of from about 2 hours to about 4 hours. In some embodiments the present invention provides kits that may be useful in carrying out the methods described herein. For example, in some embodiments the present invention provides a kit for obtaining tagged RNA from mammalian cells of interest present in a tissue or tissue culture that contains multiple mammalian cell types, the kit comprising two or more components selected from the group consisting of (a) a halogenated cytosine (for example 5-fluoro-cytosine), (b) thymine, (c) a nucleotide molecule encoding a cytosine deaminase enzyme, (d) a nucleotide molecule encoding a UPRT enzyme, and (e) an antibody that binds to halogenated uridine-tagged RNA (for example an anti-BrdU antibody).

The present invention also provides substantially pure samples of halogenated uridine-tagged RNA, such as substantially pure samples of 5-fluoro-uridine-tagged RNA. In some embodiments such samples may be produced using the methods of the present invention.

These and other embodiments of the invention are further described in the “Brief Description of the Figures,” “Detailed Description,” “Examples,” “Figures,” and “Claims” sections of this patent disclosure, each of which sections is intended to be read in conjunction with, and in the context of, all other sections of the present patent disclosure. Furthermore, one of skill in the art will recognize that the various embodiments of the present invention described herein can be combined in various different ways, and that such combinations are within the scope of the present invention.

### BRIEF DESCRIPTION OF THE FIGURES

FIG. 1a-f. Labeling and purification of RNA by CD expression and 5-FC treatment. FIG. 1a—Schematic diagram showing RNA labeling using CD and 5-FC. FIG. 1b—Chemical reactions steps involved in the labeling of RNA using CD and 5-FC. The dotted arrow indicates the intermediary steps that are not shown. FIG. 1c Relative fold enrichment of mRNA isolated by Flura-tagging. 293T cells expressing CD or unlabeled controls were treated with 5-FC for the indicated times, labeled mRNAs were immunopurified using anti-BrdU antibody, and the levels of labeled mRNAs of the indicated genes relative to corresponding non-immunoprecipitated inputs normalized to immunopurified mRNAs from cells not expressing CD were quantified by RT-PCR. The data indicate the fold enrichment over unlabeled control cells not expressing CD (n=3, ±S.E.). FIG. 1d—Schematic diagram of the constructs used for inducible expression of UPRT and/or CD, and the experimental design. Transduced RFP+MDA231 cells were mixed with unlabeled cells, treated with doxycycline for 24 h and then 5-FC for 4-12 h. Cells were analyzed by immunofluorescence or harvested for RNA analysis. FIG. 1e—Flura-tagging in MDA231 cells in vitro. MDA231 cells expressing RFP-IRES-CD or UPRT-T2A-RFP-IRES-CD were co-cultured with untransduced control cells, treated with 5-FC, and immunostained using anti-BrdU antibody (n=3). Scale bar=20 μM. FIG. 1f—100, 500 or 1,000 MDA231 cells expressing CD/UPRT were co-cultured with 10⁶ mouse 4T1 cells, treated with 5-FC for 12 h, and Flura-tagged RNAs were immunoprecipitated. The fold enrichment of the indicated representative human genes over mouse housekeeping genes (mHPRT1 and mLDH1) was measured by RT-PCR (n=3±S.E.). The left (darker gray) bar in each pair of bars is hTUBB data. The right (lighter gray) bar in each pair of bars is hHPRT1 data.

FIG. 2a-b. Flura-tagging has minimal cytotoxicity. FIG. 2a—Effect of Flura-tagging on stress granule formation. MDA231 cells were treated with 5-FC for the indicated times, and the formation of stress granules was detected by G3BP immunostaining. Control cells were either treated with water or sodium arsenite (Arsenite) for one hour. Arrows indicate stress granules (n=3). Scale bar=20 μM. FIG. 2b—Effect of Flura-tagging on transcription. RNA expression in control cells was compared to CD/UPRT-expressing cells treated with the indicated concentrations of 5-FC for 4 h or 12 h. Each dot represents a gene, with the outer/lighter gray dots indicating genes showing more than 2-fold differential expression, as determined by DESeq2 (n=2). The dotted line represents the four-fold cutoff mark.

FIG. 3 a-g. Flura-tagging of mRNA in vivo. FIG. 3a—Schematic diagram of lung colonization xenograft assay used for evaluation of Flura-tagging/Flura-seq. Athymic mice were injected through the tail vein with 50,000 MDA231 cells expressing CD/UPRT and GFP-luciferase. Upon the development of detectable bioluminescent lung metastases (4 weeks), mice were treated with doxycycline to induce CD/UPRT expression and injected with 5-FC. Lungs were harvested, and Flura-tagged mRNAs were immunopurified. FIG. 3b—Comparison of signal-to-noise ratio of Flura-tagging and TU-tagging in vivo. Mice were injected with either 5-FC or TU, the Flura-tagged or TU-tagged mRNAs were purified 12 h post injection, and the relative fold enrichment of representative human housekeeping genes relative to representative murine housekeeping genes (mHPRT1, mLDH1, mPGK1 and mGAPDH), normalized to their corresponding unpurified inputs, were measured by RT-PCR (n=5, ±S.E.). FIG. 3c—Quantification of the number of human cells in the mouse lungs used for Flura-tagging. Half of the mouse lungs used for Flura-tagging in FIG. 3b was dissociated into single cells, and the number of RFP+ cells was quantified using flow cytometry. The total number of MDA231 cells per lung was estimated based on an assumption of 1.5×10⁸ cells per mouse lung (20) (n=5). FIG. 3d—Flura-seq specifically identifies tagged human transcripts from lung micrometastases. Flura-tagged RNA from mice lungs bearing CD/UPRT expressing MDA231 and treated with 5-FC for 4 h or 12 h were immunopurified and sequenced. RNA reads were aligned to a hybrid genome containing the human and mouse genomes. The percentage of aligned reads mapped to human genome for the Flura-seq samples and the corresponding unprecipitated input is indicated (n=2). FIG. 3e—Number of human and mouse genes identified by Flura-seq (samples with 4 h 5-FC treatment) at different fold enrichment cutoffs relative to the corresponding unprecipitated inputs (n=2). Uppermost data points and line (starting at 7487 enriched genes) are human data. Lower most data points and line (starting at 231 enriched genes) are mouse data. FIG. 3f—Identification of genes that are differentially expressed in MDA231 cells in mouse lung micrometastases compared to these cells cultured in vitro. Plot of mean gene expression in vivo in lung micrometastases versus the loge fold change in the gene expression in the cells cultured in vitro. Each dot represents a gene, and the outer/lighter gray dots represent genes that showed more than 4-fold differential expression, as determined by DESeq2 (n=2). FIG. 3 g—Gene Set Enrichment Analysis (GSEA) with different signaling pathway classifiers applied to the genes that were differentially expressed in vivo vs. in vitro. Heat maps of the most differentially expressed signaling pathways are shown (n=2).

FIG. 4 a-b. CD expression produces fluorouracil (5-FU) derivatives upon fluorocytosine (5-FC) treatment that can be detected using anti-BrdU antibody. FIG. 4a—CD-expressing or control 293T cells were treated with either 5-FC or 5-FU for 24 h, followed by immunofluorescent staining with anti-BrdU antibody. Scale bar=20 μM. FIG. 4b—Schematic diagram of the steps involved in labeling RNAs using Flura-tagging. Molecules that can diffuse across the cell membranes along a concentration gradient are 5-FC, 5-FU and 5-FUR, membrane non-permeable molecules are 5-FUMP, 5-FUDP, 5-FUTP, and F-RNA. Uridine phosphorylase (UP) and Uridine kinase (UK) are enzymes expressed in mammalian cells that act on 5-FU or its derivative in the indicated reaction steps.

FIG. 5 a-e. Flura-tagging enables transcriptional profiling from micrometastatic lung xenografts. FIG. 5a—Representative H & E stained sections of mouse lungs harboring micrometastases (arrows) used in Flura-tagging experiments. Inset shows higher magnification. Scale bar=2 mm (top), 100 μM (bottom). FIG. 5b—Representative micrometastasis in the context of lung vasculature. MDA231 cells were stained with anti-GFP antibody, blood vessels with anti-CD31 antibody and nuclei (DNA) with Hoechst 33258. Scale bar=50 μM. FIG. 5c—Quantification of the number of cells per micrometastasis per tissue section in engrafted lungs of mice used in the Flura-tagging experiments. The number of nuclei in individual micrometastases in lung sections from 16 separate mice were counted. FIG. 5d—Flura-tagging has minimal effects on transcription in vivo. Mice were subjected to tail vein injection with control or CD/UPRT-expressing MDA231 cells. Expression of the indicated representative genes in the lung lysates containing control MDA231 cells or MDA231 cells expressing CD/UPRT and treated with 250 mg/kg 5-FC for 8 h, was measured by RT-PCR using human specific primers (n=3). The left (darker gray) bar in each pair of bars is CD/UPRT data. The right (lighter gray) bar in each pair of bars is control data. FIG. 5e—Flow cytometric quantification of the number of RFP+ MDA231 cells in mouse lung in the Flura-tagging experiments.

FIG. 6 a-b. Validation of differential gene expression by MDA231 cells in vitro vs. as lung micrometastases in vivo. Expression of representative differentially expressed genes identified by Flura-seq in MDA231 cells cultured in vitro or in mice lungs as micrometastases, was quantified by RT-PCR using human specific primers. FIG. 6a—Genes were identified to be up-regulated in vivo. FIG. 6b—Genes were identified to be down-regulated in vivo. The expression level was normalized to in vitro samples (n=3, ±S.E.). In both FIG. 6a and FIG. 6b the left bar in each pair of bars is in vitro data and the right bar in each pair of bars is in vivo data.

### DETAILED DESCRIPTION

Some of the main embodiments of the present invention are described in the “Summary of the Invention,” “Examples,” “Brief Description of the Figures,” and “Figures” sections of this patent disclosure. This Detailed Description section provides certain additional description and details, and is intended to be read in conjunction with all other sections of the present patent disclosure.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systeme International de Unites (SI) accepted form. Numeric ranges provided herein are inclusive of the numbers defining the range.

Where a numeric term is preceded by “about” or “approximately,” the term includes the stated number and values ±10% of the stated number.

Numbers in parentheses or superscript following text in this patent disclosure refer to the numbered references provided in the “Reference List” section at the end of this patent disclosure.

Other terms are defined elsewhere in this patent disclosure, or else are used in accordance with their usual meaning in the art.

Where the present disclosure provides specified nucleotide and/or amino acid sequences (for example as identified using SEQ ID Nos), in addition to the specific sequences that are disclosed herein, variants of such sequences are also contemplated and are intended to fall within the scope of the present invention. For example, in some embodiments variants of the specific sequences disclosed herein from other species (orthologs) may be used. Similarly, in other embodiments variants that comprise fragments of any of the specific sequences disclosed herein may be used. Likewise, in some embodiments variants of the specific sequences disclosed herein that comprise one or more substitutions, additions, deletions, or other mutations may be used. In some embodiments the variant sequences have at least about 40% or 50% or 60% or 65% or 70% or 75% or 80% or 85% or 90% or 95% or 98% or 99% identity with the specific sequences described herein.

### Halogenated Cytosine and Other Compounds

The various methods described herein require that a halogenated cytosine is contacted with the cells of interest. Any suitable halogenated derivative of cytosine may be used. In some embodiments the halogenated cytosine is selected from the group consisting of fluoro-cytosine, chloro-cytosine, bromo-cytosine, and iodo-cytosine. Furthermore, in some embodiments the halogenated cytosine is selected from the group consisting of 5-fluoro-cytosine, 5-chloro-cytosine, 5-bromo-cytosine, and 5-iodo-cytosine. In some embodiments the halogenated cytosine is 5-fluoro-cytosine. Halogenated cytosine derivatives such as those identified herein are known in the art and are either available from commercial sources or can be produced using published protocols. For example, 5-fluoro-cytosine is commercially available from Sigma-Aldrich (catalog number F7129).

Some of the methods described herein require that thymine is contacted with the cells of interest. Thymine is well known in the art and is available from multiple commercial sources and/or can be produced using published protocols. For example, thymine is commercially available from Sigma-Aldrich (catalog number T0376).

Any suitable method may be used to bring the halogenated cytosine and/or thymine into contact with the cells of interest. For example, when the cells of interest are present in vitro, such as in a tissue culture, the halogenated cytosine and/or thymine may be simply added to the tissue culture medium. When the cells of interest are present in an animal in vivo, the halogenated cytosine and/or thymine may be administered to the animal by any means that will result in the halogenated cytosine and/or thymine reaching and coming into contact with the cells of interest in that animal. For example, the halogenated cytosine and/or thymine may be administered to the animal by oral, intravenous, intraperitoneal, or subcutaneous routes, or by any other suitable route of administration known in the art. In some embodiments the agents may be administered systemically. In other embodiments the agents may be administered locally.

The amount of halogenated cytosine and/or thymine that is used should be an “effective amount.” As used herein the term “effective amount” refers to an amount of one of these agents that is sufficient to achieve the desired outcomes described herein. For example, in the case of the halogenated cytosine, the amount should be sufficient to achieve the desired outcome of “tagging” newly synthesized RNA in the cells of interest—as described herein. Similarly, in the case of thymine, the amount should be sufficient to act as a competitive inhibitor of halogenated uracil/halogenated uridine export from CD-expressing cells—as described herein. An appropriate “effective” amount of a halogenated cytosine and/or thymine may be determined using standard techniques known in the art, such as in vitro and/or in vivo dose escalation studies, and may be determined taking into account such factors as the desired route of administration, desired frequency of administration, etc. In some embodiments an “effective amount” may be determined using assays such as those described in the Examples section of this patent application. In some embodiments an “effective amount” may be calculated or determined based on studies performed in vitro and/or in vivo in various animal models, and may be determined taking into account various factors such as the form of the agent, the route of administration, the body weight of the animal, etc.

In some embodiments the halogenated cytosine used in the methods of the present invention is 5-fluoro-cytosine. The 5-fluoro-cytosine is converted in CD-expressing cells to various metabolites, including 5-fluoro-uracil (5-FU). 5-FU can be transported across cell membranes based on its concentration gradient (9,10). Furthermore, 5-FU is highly cytotoxic, and is in fact used extensively used as a chemotherapeutic agent for the treatment of solid tumors—based on its ability to induce cell death by inhibiting thymidylate synthase and by causing DNA and RNA damage (12-14). Importantly, the present invention provides various means for minimizing any such cytotoxicity while still allowing for effective RNA labeling. For example, and as described in more detail in the Examples section of this patent disclosure, the present invention provides amounts of 5-fluoro-cytosine, and timing of 5-fluoro-cytosine administration, that have been optimized to minimize cytotoxicity while facilitating effective RNA labeling. The present patent disclosure also provides specific assays that can be performed to measure such cytotoxicity.

In some embodiments the halogenated cytosine is used at approximately the maximum amount/dose at which it can be used without inducing cytotoxicity, for example as determined using the assays described in the Examples section of this patent disclosure. In some embodiments the halogenated cytosine is used at about 90% of the maximum amount/dose at which it can be used without inducing cytotoxicity. In some embodiments the halogenated cytosine is used at about 80% of the maximum amount/dose at which it can be used without inducing cytotoxicity. In some embodiments the halogenated cytosine is used at about 75% of the maximum amount/dose at which it can be used without inducing cytotoxicity. In some embodiments the halogenated cytosine is used at about 70% of the maximum amount/dose at which it can be used without inducing cytotoxicity. In some embodiments the halogenated cytosine is used at about 60% of the maximum amount/dose at which it can be used without inducing cytotoxicity. In some embodiments the halogenated cytosine is used at about 50% of the maximum amount/dose at which it can be used without inducing cytotoxicity. In some embodiments the halogenated cytosine is used at about 40% of the maximum amount/dose at which it can be used without inducing cytotoxicity. In some embodiments the halogenated cytosine is used at about 30% of the maximum amount/dose at which it can be used without inducing cytotoxicity. In some embodiments the halogenated cytosine is used at about 25% of the maximum amount/dose at which it can be used without inducing cytotoxicity. In some embodiments the halogenated cytosine is used at about 20% of the maximum amount/dose at which it can be used without inducing cytotoxicity. In some embodiments the halogenated cytosine is used at about 10% of the maximum amount/dose at which it can be used without inducing cytotoxicity. In some embodiments the halogenated cytosine is used at about 5% of the maximum amount/dose at which it can be used without inducing cytotoxicity. In some embodiments the halogenated cytosine is used at about 1% of the maximum amount/dose at which it can be used without inducing cytotoxicity.

In some embodiments the cells of interest are contacted with a halogenated cytosine in vitro. In some such embodiments, the effective amount of the halogenated cytosine is up to about 50 micro-molar, or up to about 100 micro-molar, or up to about 150 micro-molar, or up to about 200 micro-molar, or up to about 250 micro molar, or up to about 300 micro molar, or up to about 350 micro molar, or up to about 400 micro molar, or up to about 450 micro molar, or up to about 500 micro molar. In other some such embodiments, the effective amount of the halogenated cytosine is about 50 micro-molar, or about 100 micro-molar, or about 150 micro-molar, or about 200 micro-molar, or about 250 micro molar, or about 300 micro molar, or about 350 micro molar, or about 400 micro molar, or about 450 micro molar, or about 500 micro molar.

In some embodiments the cells of interest are contacted with a halogenated cytosine in vivo. In some such embodiments, the effective amount of the halogenated cytosine is up to about 50 mg/kg, or up to about 100 mg/kg, or up to about 150 mg/kg, or up to about 200 mg/kg, or up to about 250 mg/kg, or up to about 300 mg/kg, or up to about 350 mg/kg, or up to about 400 mg/kg, or up to about 450 mg/kg, or up to about 500 mg/kg. In some such embodiments, the effective amount of the halogenated cytosine is about 50 mg/kg, or about 100 mg/kg, or about 150 mg/kg, or about 200 mg/kg, or about 250 mg/kg, or about 300 mg/kg, or about 350 mg/kg, or about 400 mg/kg, or about 450 mg/kg, or about 500 mg/kg.

In some embodiments the cells of interest are contacted with thymine in vitro. In some such embodiments, the effective amount of the thymine is up to about 50 micro-molar, or up to about 100 micro-molar, or up to about 125 micro-molar, or up to about 150 micro-molar, or up to about 200 micro-molar, or up to about 250 micro molar, or up to about 300 micro molar, or up to about 350 micro molar, or up to about 400 micro molar, or up to about 450 micro molar, or up to about 500 micro molar. In other some such embodiments, the effective amount of the thymine is about 50 micro-molar, or about 100 micro-molar, or about 125 micro-molar, or about 150 micro-molar, or about 200 micro-molar, or about 250 micro molar, or about 300 micro molar, or about 350 micro molar, or about 400 micro molar, or about 450 micro molar, or about 500 micro molar.

In some embodiments the cells of interest are contacted with thymine in vivo. In some such embodiments, the effective amount of the thymine is up to about 50 mg/kg, or up to about 100 mg/kg, or up to about 125 mg/kg, or up to about 150 mg/kg, or up to about 200 mg/kg, or up to about 250 mg/kg, or up to about 300 mg/kg, or up to about 350 mg/kg, or up to about 400 mg/kg, or up to about 450 mg/kg, or up to about 500 mg/kg. In some such embodiments, the effective amount of the thymine is about 50 mg/kg, or about 100 mg/kg, or about 125 mg/kg, or about 150 mg/kg, or about 200 mg/kg, or about 250 mg/kg, or about 300 mg/kg, or about 350 mg/kg, or about 400 mg/kg, or about 450 mg/kg, or about 500 mg/kg.

In some embodiments the tissue or tissue culture is contacted with the halogenated cytosine and/or the thymine in vitro or in vivo for a period of at least about 2 hours, and for a period of up to about 4 hours, or up to about 6 hours, or up to about 8 hours, or up to about 10 hours, or up to about 12 hours, or up to about 24 hours, or up to about 48 hours. In some embodiments the tissue or tissue culture is contacted with a halogenated cytosine and with thymine concurrently.

### Enzymes, & Vectors for Expression Thereof

The CD enzyme is a prokaryotic and fungal enzyme not expressed by mammalian cells. The methods of the present invention involve expression of a recombinant cytosine deaminase (CD) enzyme in cells of interest. In some embodiments the cells of interest are not fungi and are not prokaryotic cells. In some embodiment the cells of interest are mammalian cells.

Any suitable CD enzyme, from any suitable species, can be used as long as the enzyme has cytosine deaminase activity in the cells of interest. Similarly, any naturally occurring or manmade variant of a CD enzyme can be used as long as the enzyme has cytosine deaminase in the cells of interest. In the Examples presented herein the CD enzyme used was from Saccharomyces cerevisiae. However, a CD enzyme from any other organism or species, or a variant thereof, can be used as long as the enzyme has cytosine deaminase activity in the cells of interest. Nucleotide sequences and amino acid sequences of CD enzymes are known in the art and provided in public nucleotide and amino acid sequence databases. Furthermore, vectors comprising nucleotide sequences encoding CD enzymes are available commercially. For example, a vector comprising a nucleotide sequence encoding CD is commercially available from Addgene (catalog number 35102).

Some of the methods of the present invention involve expression of a recombinant uracil phosphoribosyltransferase (UPRT) enzyme in the cells of interest. Any suitable UPRT enzyme can be used as long as the enzyme has uracil phosphoribosyltransferase activity in the cells of interest. Similarly, any naturally occurring or manmade variant of a UPRT enzyme can be used as long as the enzyme has uracil phosphoribosyltransferase activity in the cells of interest. In the Examples presented herein the UPRT enzyme used was from Toxoplasma gondii. However, a UPRT enzyme from any other organism or species, or a variant thereof, can be used as long as the enzyme has UPRT activity in the cells of interest.

Nucleotide sequences and amino acid sequences of UPRT enzymes are known in the art and provided in public nucleotide and amino acid sequence databases. Furthermore, vectors comprising nucleotide sequences encoding UPRT enzymes are available commercially. For example, a vector comprising a nucleotide sequence encoding UPRT is available from Addgene (catalog number 47110).

The recombinant enzymes used in the methods of the present invention can be expressed in the cells of interest using any suitable vector system and any suitable promoter system known in the art. For example, the recombinant CD and/or UPRT enzymes can be expressed in the cells of interest by delivering to the cells, or by obtaining cells that already comprise, a recombinant nucleic acid molecule that comprises a nucleotide sequence encoding the enzyme (i.e. CD or UPRT) operatively linked to a promoter. Some embodiments involve delivering to the cells of interest, or obtaining cells of interest that already comprise, both a recombinant nucleotide sequence encoding a CD enzyme and a recombinant nucleotide sequence encoding a UPRT enzyme. In some such embodiments the nucleotide sequence encoding the CD enzyme and the nucleotide sequence encoding the UPRT enzyme are present on the same nucleic acid molecule. In some such embodiments the nucleic acid molecule comprises an internal ribosome entry site (IRES) sequence or viral 2A peptide encoding sequence located between the nucleotide sequence encoding the CD enzyme and the nucleotide sequence encoding the UPRT enzyme. In other embodiments the nucleotide sequence encoding the CD enzyme and the nucleotide sequence encoding the UPRT enzyme are each present on a separate nucleic acid molecule.

The promoter(s) used to drive expression of the recombinant CD and/or UPRT enzymes can be any promoters known in the art that allow for expression of the enzyme(s) in the cells of interest. In some embodiments the promoter is an inducible promoter. Any suitable inducible promoter system known in the art can be used. Exemplary inducible promoter systems include, but are not limited to, doxycycline (Dox)-inducible promoters.

In some embodiments the promoter is a tissue-specific promoter, or other cell-type specific promoter, or the promoter activity is regulated by cell-type or tissue specific expression of recombinase such as, Cre recombinase, for example to drive expression specifically in the cells of interest. Any suitable tissue-specific promoter system known in the art can be used.

Methods for delivery of vectors or other nucleic acid molecules to cells either in vitro or in vivo are well known in the art, and any suitable methods can be used in conjunction with the present invention. In some embodiments of the present invention the nucleic acid molecules described herein are delivered to cells in vitro. In other embodiments of the present invention the nucleic acid molecules of the invention are delivered to cells in vivo. In some embodiments of the present invention the nucleic acid molecules described herein are delivered to cells in vitro and the cells are then delivered to an animal. In some embodiments the nucleic acid molecules described herein are present and/or expressed in the cells of interest transiently. In some embodiments the nucleic acid molecules described herein are present and/or expressed in the cells of interest stably/permanently. In some embodiments the nucleic acid molecules described herein are used to generate transgenic animals containing the recombinant CD and/or UPRT sequences. The vector to be used, and the means used for delivery of that vector, can be selected based on the situation, e.g. the cell type, whether stable or transient transfection/transduction is required, whether an integrating vector is desired, whether the cells of interest are in vivo or in vitro, and the like.

### Antibodies & Immuno-Affinity Purification Methods

One of the major advantages of the methods of the present invention is that the tagged halogenated-uridine-containing RNAs can be detected and isolated/purified using simple and widely available technologies—employing antibodies that bind to halogenated-uridine-containing RNAs, but not halogenated-cytidine-containing RNAs, and routine immuno-affinity purification techniques. In some embodiments, the halogenated-uridine-containing RNAs technologies can be detected using any antibody known in the art that binds specifically to halogenated-uridine-containing RNAs but that does not bind to non-halogenated RNAs. One such type of antibody is an anti-BrdU antibody, of which several are known in the art and available from commercial sources, including but not limited to, the anti-BrdU antibody available from Abcam (catalog number ab6326). Any routine immuno-affinity purification techniques can be used to the tagged halogenated-uridine-containing RNAs, including, but not limited to immuno-affinity chromatography methods, including column-based methods, bead-based methods, and the like. For example, standard immno-precipitation techniques can be used. In some embodiments, two or more successive rounds of immuno-affinity purification are used.

### Transcriptome Analysis

Tagged halogenated-uridine-containing RNAs obtained from cells of interest using the methods of the present invention can be analyzed in any way that any other RNA can be analyzed. Such methods include, but are not limited to, performing hybridization-based analysis (such as microarray analysis), RT-PCR analysis, next generation RNA sequencing (“RNA-seq”) analysis, and the like. Methods of performing such analyses are well known in the art and described in the published literature. Furthermore, kits for performing such analysis, including kits for RNA-seq library preparation and sequencing, are commercially available from multiple sources.

Embodiments of the present invention can be further described and understood by reference to the following non-limiting “Example.” It will be apparent to those skilled in the art that many modifications to the specific description provided in the Example can be practiced without undue experimentation and without departing from the spirit of and scope of the present invention.

### Example

Numerous biologically and clinically significant cell populations represent only minute fractions of tissues, however, existing techniques lack the sensitivity to faithfully capture rare transcriptomes in situ (1-3). To overcome this problem, we developed fluorouracil-tagged RNA sequencing (Flura-seq) to define the transcriptomes of rare cells of interest from an intact tissue microenvironment. Fluraseq utilizes cytosine deaminase (CD) to convert the non-natural pyrimidine fluorocytosine to fluorouracil. Expression of S. cerevisiae CD and exposure to fluorocytosine generates fluorouracil and metabolically labels newly synthesized RNAs specifically in cells of interest. Fluorouracil-tagged RNAs can then be immunopurified and sequenced. We applied Flura-seq to define the transcriptome of human breast cancer xenografts representing as few as 0.003% of host organ cell population during the early stages of metastatic colonization of mouse lungs. The robustness, simplicity and lack of toxicity of Flura-seq make this tool broadly applicable to many studies in developmental, regenerative, and cancer biology.

Tissues are comprised of different cell types whose interactions elicit distinct gene expression patterns that regulate tissue formation, regeneration, homeostasis and repair. Analysis of these gene expression patterns require methods that can capture as closely as possible the transcriptomes of cells of interest in their tissue microenvironment. Current technologies designed to study in situ transcriptomics are limited by their low sensitivity (requiring more than 1% of the total tissue) (1-3), the involvement of multiple steps after tissue dissociation (3-5), or the requirement for sophisticated tools (6), making it challenging to transcriptionally profile rare cell populations rapidly isolated from their native microenvironment. To address this problem, here we describe the development of Flura-seq and its initial application to the analysis of micrometastatic cell populations representing a tiny fraction of the host organ. Flura-seq is based on the use of cytosine deaminase (CD), a key enzyme of the pyrimidine salvage pathway in fungi and prokaryotes (7)′ CD is absent in mammalian cells, which instead use cytidine deaminase for the same purpose (7). In addition to converting cytosine to uracil, CD can also convert 5-fluorocytosine (5-FC), a non-natural pyrimidine, to 5-flourouracil (5-FU). 5-FU is endogenously converted to fluorouridine triphosphate (F-UTP), which is then incorporated into RNA.

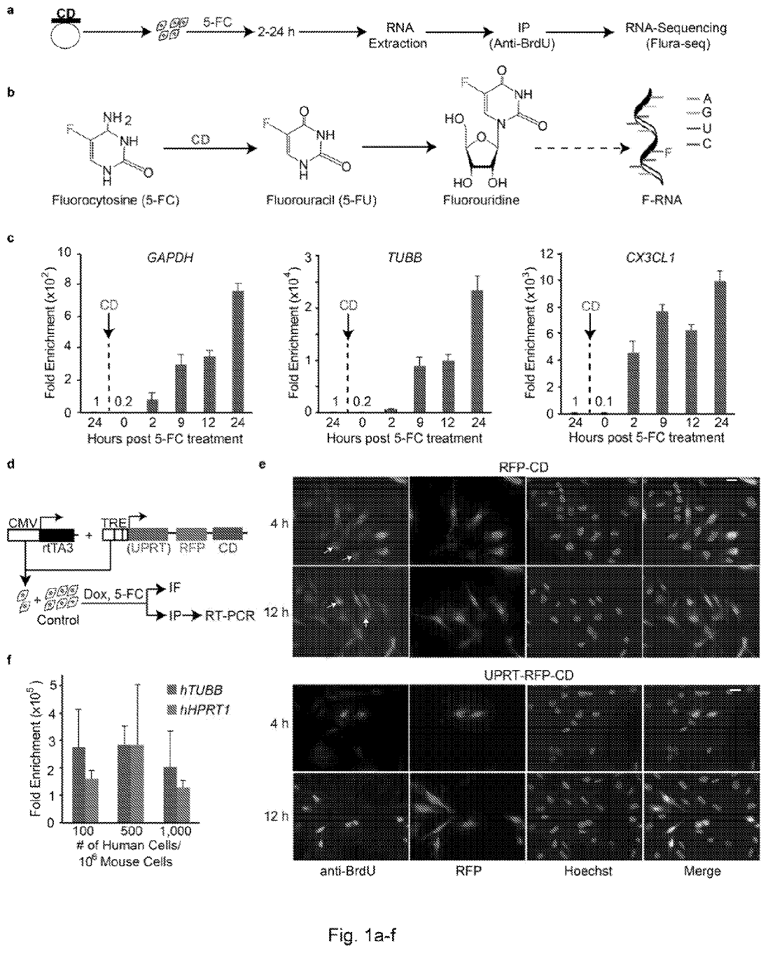

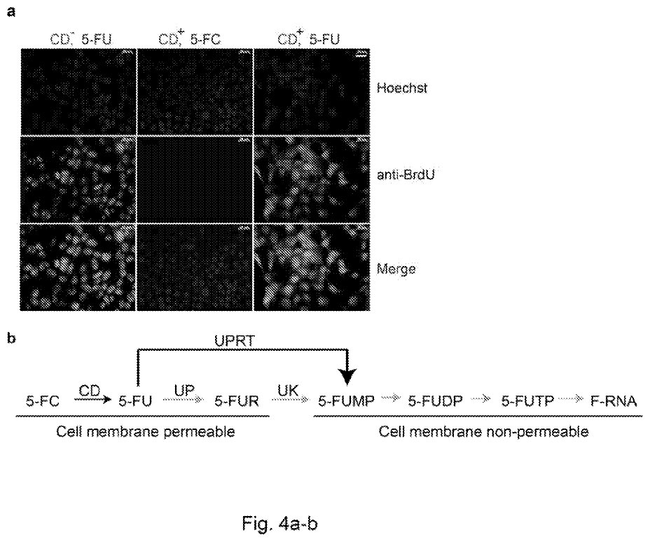

S. cerevisiae CD was exogenously expressed in human embryonic kidney 293T cells, and the cells were treated with 250 μM fluorocytosine (5-FC). This system is predicted to generate intracellular fluorouracil (5-FU), which is then incorporated into newly synthesized RNAs (FIG. 1a, b). Antibodies against bromodeoxyuridine (BrdU) can recognize BrdU and other halogenated uridines incorporated into nucleic acids (8). Indeed, untransfected control cells incubated with 5-FU showed positive immunostaining with anti-BrdU antibody, whereas cells incubated with 5-FC did not, suggesting that the antibody specifically interacts with 5-FU but not 5-FC derivatives (FIG. 4a). As anticipated, cells expressing CD were stained by the antibody when treated with 5-FC (FIG. 4a).

Next, we determined whether 5-FU-labeled mRNAs can be specifically and efficiently isolated by immunoprecipitation. Labeled mRNAs of three representative genes with varying levels of expression were detectable as early as 2 h after treatment with 5-FC, and the labeling continued to increase for up to 24 h (FIG. 1c). When the mRNAs were immunoprecipitated, released and re-precipitated, the signal from the negative control sample not expressing CD was undetectable whereas the enrichment of the 5-FU-tagged RNAs was not significantly affected (data not shown). Therefore, in subsequent experiments, the RNAs were immunoprecipitated, released and re-precipitated to further reduce the background of unlabeled RNAs. Collectively, these results demonstrate that Flura-tagging allows highly specific labeling and purification of newly synthesized transcripts. 5-FU can be transported across cell membranes based on its concentration gradient (9,10). Therefore, we determined whether 5-FU generated in CD-expressing cells could diffuse to neighboring cells that do not express the enzyme by co-culturing CD-expressing human breast cancer MDA-MB-231 cells (abbreviated MDA231) with unlabeled control cells (FIG. 1d). Treatment with 5-FC led to 5-FU-labeling of cells that did not express CD as well (FIG. 1e). In order to prevent the diffusion of 5-FU from CD-expressing cells, we implemented a dual strategy. First, we engineered CD-bearing cells to co-express Uracil phosphoribosyl transferase (UPRT), facilitating the conversion of 5-FU to F-UMP, which does not diffuse across cell membranes. UPRT expression bypasses the conversion of 5-FU to FUR, both of which can be transported across cell membranes along the concentration gradient, and thus reduces the intracellular level of 5-FU (FIG. 4b). To this end, we developed a polycistronic vector that allows doxycycline (Dox)-inducible co-expression of CD, UPRT and RFP (referred as CD/UPRT hereafter) (FIG. 1e). Second, since thymine can competitively inhibit cellular uptake of 5-FU (11), we included thymine in the medium as a competitive inhibitor of 5-FU export from CD-expressing cells. This dual strategy restricted the anti-BrdU immunostaining to cells expressing CD (FIG. 1e). In subsequent in vitro and in vivo experiments, thymine was used along with 5-FC.

Next, we determined whether this system could be used to isolate RNA specifically from cells of interest that were admixed with a large proportion of other cells. MDA231 cells expressing CD/UPRT were co-cultured with the mouse breast cancer 4T1 cell line at ratios of 10′ to 10′ (100-1000 MDA231 cells to 10⁶ 4T1 cells). After 12 h of 5-FC addition, 5-FU-labeled mRNAs were immunoprecipitated, and the proportion of human and mouse mRNA for representative housekeeping genes were determined by reverse transcriptase-polymerase chain reaction (RT-PCR). Human mRNAs were enriched by more than 10⁵ fold compared to mouse mRNAs (FIG. 10, demonstrating the efficacy and specificity of the technique in measuring newly synthesized RNAs from small cell populations of interest in a heterogeneous mixture of cells.

5-FU is extensively used as a chemotherapeutic agent for the treatment of solid tumors, based on its ability to induce cancer cell death by inhibiting thymidylate synthase and by causing DNA and RNA damage (12-14). It was therefore crucial to test whether our 5-FU-tagging method, which is based on low concentrations of 5-FC and short incubation time periods, has significant cytotoxic effects under our experimental conditions. CD/UPRT-expressing cells were treated with 5-FC at concentrations of up to 250 μM for up to 12 h (“Flura-tagged” cells), and the effects of this treatment on RNA damage response and transcriptional regulation were investigated.

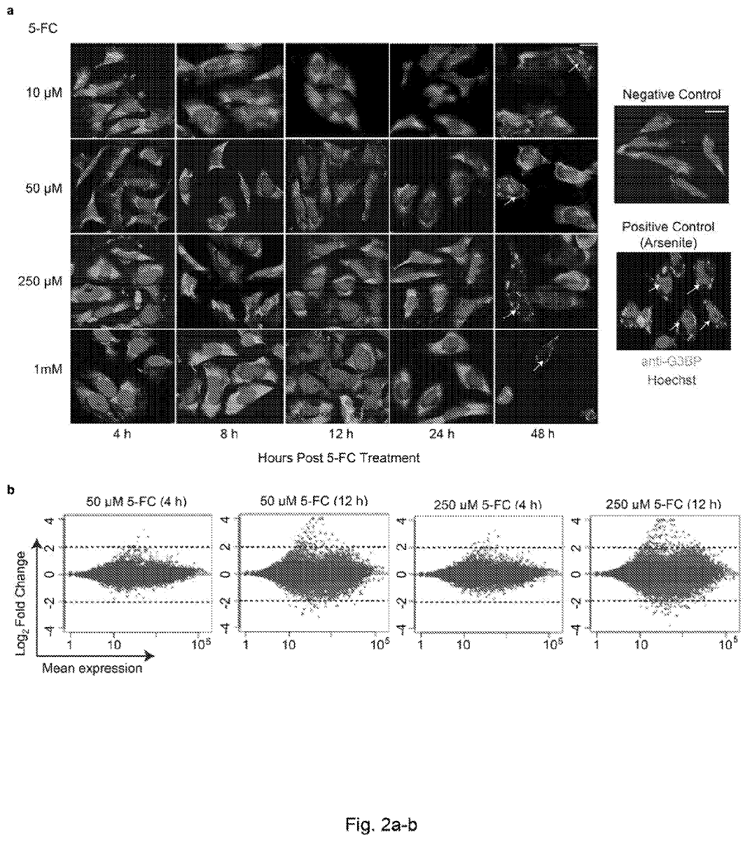

A key feature of the RNA damage response is the formation of stress granules (15). Ras GTPase-activating protein-binding protein 1 (G3BP), a key component of stress granules, forms distinct foci during stress granule formation (12). We determined the dose- and time-dependent effect of 5-FC treatment on stress granule formation by immunostaining of the cells with anti-G3BP antibody. Cells treated with sodium arsenite (NaAsO₂), which causes stress granule formation (12), formed distinct G3BP foci within 1 h, whereas cells treated with 5-FC did not contain stress granules after 12 h with any of the tested concentration (FIG. 2a). The 5-FC-treated cells started to form stress granules only after 24 h of treatment with 1 mM 5-FC, a concentration four times higher than the maximal concentration of 5-FC used for Flura-tagging, or after 48 h of 5-FC treatment at lower concentrations (FIG. 2a). Based on these results, we set 250 μM 5-FC and 12 h as the upper limits for Flura-tagging of these cells.

For a more sensitive analysis of potential alterations caused by Flura-tagging, we investigated the transcriptional changes occurring under these experimental conditions. We compared the transcriptome of cells treated with two different concentrations of 5-FC (50 μM and 250 μM) at two different time points (4 h and 12 h) with that of control cells, using RNA-seq. Only 113 genes showed statistically significant differences in expression in cells treated with 50 μM of 5-FC for 4 h when compared to the control cells (FIG. 2b). This number increased to 190, 1325 and 1965 genes in cells treated with 250 μM 5-FC for 4 h, 50 μM 5-FC for 12 h, and 250 μM 5-FC for 12 h, respectively (FIG. 2b). Moreover, a majority of these differences in gene expression were within a four-fold range (FIG. 2b). Only 10, 15, 102 and 182 genes showed more than 4-fold difference with 50 μM 5-FC for 4 h, 250 μM 5-FC for 4 h, 50 μM 5-FC for 12 h and 250 μM 5-FC for 12 h, respectively. These results demonstrate that Flura-tagging introduces minimal alteration in the basal transcriptomes of cells, particularly at short (<4 h) treatment durations.

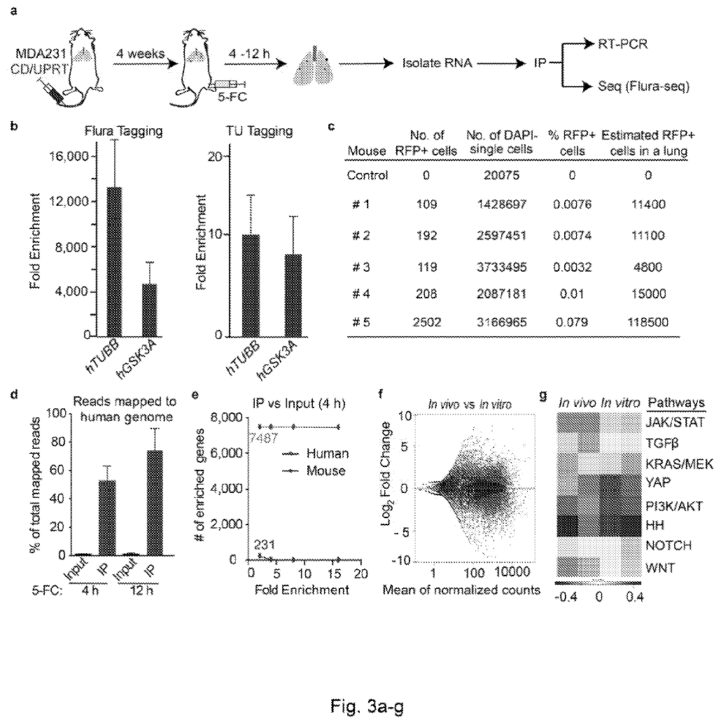

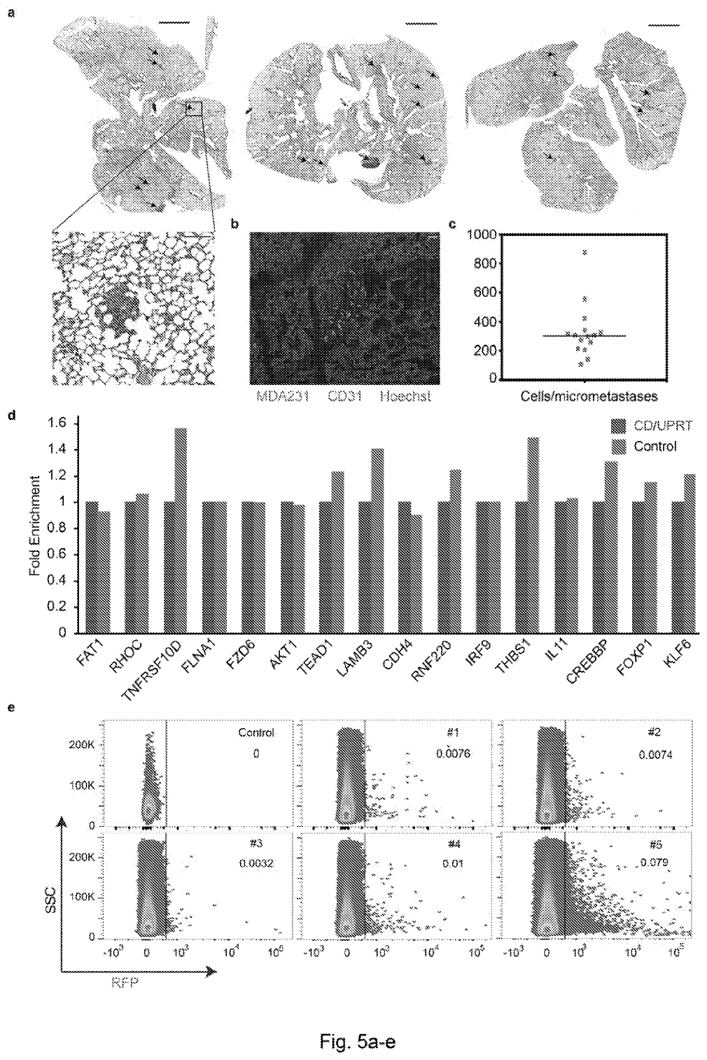

Next, we determined whether Flura-seq could be used to characterize transcriptomics in situ from a small number of cancer cells disseminated in an intact organ that would be challenging to achieve using existing technologies. Understanding the gene expression in the early stage micrometastatic cancer cells has a huge clinical significance as it represents the state of cancer cells before they develop into life threatening and difficult to treat macrometastases. MDA231 cells engineered to express a GFP-luciferase fusion protein for imaging and bioluminescence analysis, and DOX-inducible CD/UPRT for Flura-tagging of RNA, were inoculated into the tail vein of Foxn1^{nu} immunodeficient mice to allow colonization of the pulmonary parenchyma (FIG. 3a). A small proportion of the injected cells survived in the lungs and initiated metastatic outgrowth. At day 28 after inoculation, the cancer cell population was present as micrometastatic colonies throughout the pulmonary parenchyma (FIG. 5a, b). In 2-dimensional sections, the size distribution of these colonies was 112 to 877 cells per micrometastasis with a mean value of 333 cells (FIG. c5). CD/UPRT expression was induced by doxycycline treatment on day 28, and then mice were administered 5-FC (250 mg/kg) and thymine (125 mg/kg) for 4 h to 12 h before harvesting the lungs to extract Flura-tagged RNAs by immunoprecipitation (FIG. 3a).

To establish a 5-FC dose for use in vivo, we used the same dose as thiouracil (250 mg/kg) that has been established to be non-toxic for the treatment up to 12 h in mice used for tagging RNA with thiouracil, which is structurally similar to 5-FU (16). Pharmacokinetics studies have shown that 5-FC has a very short half-life in mice (0.36 to 0.43 h) (17), suggesting that large fraction of 5-FC is cleared from the body rapidly. Furthermore, the expression of several representative human genes was within 2-fold range in mouse lungs inoculated with control MDA231 cells or the cells expressing CD/UPRT and treated with 250 mg/kg 5-FC for 8 h (FIG. 5d), suggesting that the 5-FC treatment in our experimental conditions has minimal effect on gene expression in CD/UPRT-expressing MDA231 cells in vivo.

Next, we investigated the signal-to-noise ratio of Flura-tagging in vivo by measuring the relative capture of two representative housekeeping human transcripts (signal) and mouse transcripts (noise). Human mRNAs were enriched more than a 1000-fold compared to the equivalent mouse mRNAs (FIG. 3b), indicating that Flura-tagging occurs primarily in the cells of interest and that tagged RNAs can be purified efficiently from intact lung tissue. We also compared the signal-to-noise ratio of Flura-tagging with TU-tagging, an analogous covalent RNA labeling technique (18). TU tagging has been reported to work well when mice are treated with TU for up to 12 h (16), so we treated the mice for 12 h in this experiment. Analysis of tested human mRNAs compared to the mouse mRNAs showed approximately 10-fold enrichment with TU tagging compared to over 1000-fold enrichment with Flura-tagging under similar conditions (FIG. 3b). This result demonstrates that Flura-tagging has a superior signal-to-noise ratio compared to TU-tagging, likely primarily due to the inability of the cells to convert 5-FC to FU without exogenous CD expression.

To determine the sensitivity of Flura-tagging, we estimated the percentage of human cells expressing RFP (along with CD and UPRT) (FIG. 1d) in the mouse lungs in parallel experiments. We found that roughly 0.003 to 0.08% of the total cells were RFP positive human cells (FIG. 3c, FIG. 5e). Since one mouse lung contains approximately 150 million cells (19), we estimate that RNA from as few as approximately 5000 human cancer cells per mouse lung could be detected by Flura-tagging (FIG. 3c). Collectively these results indicate that Flura-tagging is efficient to capture transcripts from very rare cells in an intact organ.

Finally, we determined whether Flura-tagged mRNA from micrometastatic lesions could be sequenced to characterize the in situ transcriptomes of cancer cells. Mice were treated with 5-FC for 4 h or 12 h, and Flura-tagged RNAs were immunopurified and sequenced. The sequenced reads were aligned to a hybrid genome containing both human and mouse genomes, so that reads coming from human or mouse cells could be distinctly identified. In mice treated with 5-FC for 4 h, approximately 53% of the aligned reads were mapped to human genome, whereas 74% of the aligned reads were mapped to human genome when the mice were treated with 5-FC for 12 h (FIG. 3d). Less than 1% of the mapped reads in the non-immunopurified input samples were aligned to the human genome while 99% of the reads aligned to the mouse genome (FIG. 3d). We further sought an alternative means of distinguishing transcripts derived from the cells of interest (human cells) and other cells (mouse cells) to enable the future application of Flura-tagging to syngeneic models. To specifically enrich the transcripts from CD/UPRT expressing cells, we applied an enrichment filter to identify only those transcripts that were enriched more than two-fold relative to their corresponding unpurified inputs for further analysis. After applying the two-fold enrichment cut-off, the reads were aligned to 7487 human genes and 231 mouse genes (FIG. 3e). When the cutoffs were increased to 4, 8 and 16-fold, there was an insignificant effect on the number of human genes identified whereas the number of mouse genes either decreased significantly or was completely eliminated (FIG. 3e). These results indicate that Flura-seq can identify cell-specific transcripts by using stringent mRNA enrichment cutoffs.

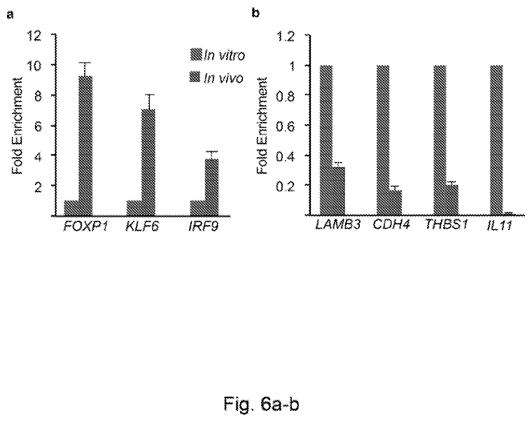

To determine the effect of in vivo growth on MDA231 lung xenografts, we compared the transcriptomic profiles of Flura-tagged MDA231 micrometastases in vivo and in tissue culture with complete growth media. The expression of 1917 genes were found to differ by more than 4-fold in vivo compared to in vitro (FIG. 3f). Up-regulation and down-regulation of some of the genes were verified by RT-PCR using human specific primers (FIG. 6a, b). Further analysis of the differentially expressed genes using classifiers of transcriptional programs downstream of specific pathways showed that the JAK/STAT, TGFβ and RAS/MEK signaling pathways were up-regulated in vivo whereas the Hippo (active YAP), PI3K, and hedgehog pathways were down-regulated (FIG. 3g). We note that the lower activity of proliferation and growth-related pathways (PI3K/AKT and YAP) relative to in vitro conditions likely reflects the high levels of growth factors supplied by the 10% fetal bovine serum supplementation of the culture media, and the rigid and highly adherent surface of tissue culture plastic vessels.

In this Example, we describe a novel method that can define in situ transcriptomes from a very small cell population representing a tiny fraction of an organ. This technique requires the expression of an exogenous enzyme to selectively label RNA in a highly controlled manner, thus minimizing noise drastically and thus making it possible to study cell types that represent a minute fraction of the total tissue. Flura-seq can be easily applied to other areas of biomedical research in model systems. For example, genetically engineered conditional expression of CD/UPRT would allow the analysis of cell-type specific transcriptomics in mice with high sensitivity. Prominent examples of specific cell types that constitute rare subpopulations within their tissues and could be targeted for analysis by Flura-seq include adult stem cells, and specific subtypes of immune and neuronal cells. Flura-seq can be applied to identify gene expression changes in these cells under different physiological, developmental and pathological conditions to uncover mechanisms involved in tissue homeostasis, regeneration and pathophysiology. Another feature of Flura-seq is that it only identifies newly synthesized transcripts, making it a powerful tool for studying changes in transcription under different stimuli, such as cytokines, pharmacologic agonists and antagonists, stress signals, and other inputs that act by rapidly changing the transcriptomic state of target cells both in vivo and in vitro. Further, since Flura-seq involves covalently labeling RNA, it can easily complement other techniques such as single-cell sequencing to combine in situ transcriptomic analysis with profiling of the dissociated cell population with single cell resolution. Thus, Flura-seq can be applied to study gene expression in rare cell populations in their native environments to address a wide range of biological questions.

### Materials & Methods

### Cell Culture

Human embryonic kidney transformed with T-cell antigen (293T) and human breast cancer MDA-MB-231 (MDA231) cells were cultured in DMEM High Glucose medium (Wheaton) supplemented with 10% Fetal Bovine Serum (Clontech, Catalog number 631106) supplemented with 2 mM L-Glutamine. All cell lines have been routinely tested for mycoplasma contamination. For the induction of CD or CD and UPRT, cells were treated with 1 μg/ml doxycycline for 24 h. For Fluorouracil tagging, cells were treated with 250 μM of 5-FC or 5-FU unless indicated. Where indicated, 125 μM thymine was added together with 5-FC. For the formation of stress granules, cells were treated with 500 μM Sodium Arsenite (Santa Cruz Biotechnologies, Catalog number SC-301816).

### Chemicals, Drugs and Antibodies

Doxycycline (Sigmal-Aldrich, Catalog number D9891), Fluorocytosine (Sigma-Aldrich, Catalog number F7129), Fluorouracil (Sigma-Aldrich, Catalog number F6627), Sodium Arsenite (Santa Cruz Biotechnology, Catalog number SC-301816), Thymine (Sigma-Aldrich, Catalog number T0376). Anti-BrdU antibody (Abcam, Catalog number ab6326), Anti-G3BP antibody (Abcam, Catalog number ab56574), Anti-CD31 antibody (Dianova, Catalog number DIA-310), Anti-GFP antibody (Ayes Labs, Catalog number GFP-1020).

### Immunofluorescence

Cells were fixed with 4% paraformaldehyde for 10 min, permeabilized with 0.2% TritonX-100 for 10 min, blocked with 5% BSA for one hour at room temperature, prior to incubation with primary antibodies at 4° C. overnight, and secondary antibodies (Thermo Fisher anti-chicken antibody, Catalog number A-11039, Thermo Fisher anti-rat antibody, Catalog number A-11006, Abcam anti-rat antibody, Catalog number ab150117) incubated for 1 h at room temperature. Mouse lungs were fixed in 4% paraformaldehyde overnight at 4° C., embedded in paraffin and sectioned. The lung sections were deparaffinized and stained with hematolxylin/eosin or immunostained using standard protocols. Automated image analysis was performed using the FIJI software package.

Flura-Tagged and TU-Tagged mRNA Extraction

Cells or tissues were lysed in lysis buffer (20 mM Tris-HCl pH 7.5, 500 mM LiCl, 1% LiDS, 1 mM EDTA, 5 mM DTT), and mRNAs were extracted using Oligo (dT)25 magnetic beads (New England Biolabs, Catalog number S1419S) following the manufacturer's recommended protocol. The isolated mRNAs were immunoprecipitated using anti-BrdU antibody (1-5 μg/sample) conjugated with Protein G Dynabeads (Thermo Fisher Scientific, Catalog number 10004D) by overnight incubation at 4° C. The mRNAs were incubated with the antibody bead complex in 0.8× Binding buffer (0.5×SSPE with 0.025% Tween 20) at room temperature for 1-2 h in a rotator. Subsequently, beads were washed twice with Binding buffer, twice with Wash buffer B (1×SSPE with 0.05% Tween 20), once with Wash buffer C (TE with 0.05% Tween 20), and once with TE buffer. The bound mRNAs were eluted in 200 μl of 100 μg/mL BrdU for 45 min in a shaker at room temperature. The eluted RNAs were purified using the RNeasy MinElute Clean up kit (Qiagen, Catalog number 74204) following the manufacturer's protocol. The RNA was eluted in 100 μl RNAase free water. The Flura-tagged RNA elute were re-precipitated as described above, and eluted in 12.5 μl final volume. The RNA was either reverse-transcribed using cDNA kit-First Strand Transcriptor (Roche, Catalog number 043790-12001) following the manufacturer's protocol, or used for Flura-Seq. TU-tagged mRNAs were purified as described in (20).

### Animal Experiments

Mouse experiments were performed following the protocols approved by the MSKCC Institutional Animal Care and Use Committee (IACUC). 5-6 weeks old female mice (Mus musculus) Hsd:Athymic-Foxnlnu were used in all the experiments. For lung colonization experiments, 50,000 MDA231 cells suspended in 100 μl PBS were injected into the tail vein. Proliferation of injected cancer cells were quantified using bioluminescence imaging following retroorbital injection of luciferin. CD/UPRT were induced by feeding mice with doxycycline containing diet for 2-3 days. For Flura-tagging, mice were injected with 250 mg/kg (500 μl) 5-FC intraperitoneally together with 125 mg/kg (500 μl) thymine subcutaneously. For thiouracil-tagging, mice were injected intraperitoneally with 250 mg/kg (500 μl) of thiouracil. The mice were euthanized 4-12 h post injection, lungs were harvested and processed for downstream experiments. For RNA analysis, lungs were dissociated using the PRO 200 grinder from PRO Scientific Inc. in RNA extraction lysis buffer. The lung lysates were either used immediately for mRNA extraction or stored at −80° C. for later use. Flura-tagged mRNAs were isolated as described above.

### Flow Cytometry

Harvested lungs were chopped into small pieces, which were then incubated at 37° C. in 30 mL digestion buffer (5% Fetal Bovine Serum (FBS) 1 mM L-glutamine 0.35 mg/mL Worthington Type III collagenase, 6.25×10-3 U/mL dispase, 100 U/mL penicillin, 100 μg/mL streptomycin, 6.25 ng/mL amphotericin B) containing 10 mL trypsin and 30 μl DNAse for 1 h. The cells were filtered through a 70 μM filter, and cells were collected by centrifugation. The cell pellets were then resuspended in PBS containing 0.1% FBS and 100 μg/ml DAPI, and analyzed using a BD FACS Aria™ IIU Flow cytometer. CD or CD/UPRT expressing stable cell lines were treated with 1 μg/mL doxycycline for 24 h, trypsinized, filtered and sorted for RFP positive cells using a BD LSRFortessa Flow cytometer.

### RNA Sequencing

RNA-seq library preparation. Total RNA was purified using Qiagen RNeasy Mini Kit. Quality and quantity was checked by Agilent BioAnalyzer 2000. 10 ng RNA per sample was used for library construction with Sample Prep Kit v2 (Illumina) according to manufacturer's instructions. Libraries were multiplex sequenced libraries on a Hiseq2500 platform, and more than 25 million raw paired-end reads were generated for each sample.

Flura-seq library preparation. RNA was amplified by SMARTer PCR with the number of PCR cycles determined empirically based on the amount of purified Flura-tagged RNA. The Nextera XT kit was used to prepare sequencing libraries following the manufacturer's protocol. In our in vivo experiments, 20 cycles of PCR were used.

### Statistics and Data Analysis

In all relevant experiments, mice were chosen randomly for different treatments. Comparisons between samples were done in the gene expression analysis, and each group had 2-3 biological replicates that are indicated in the figure legends for each experiment. The numbers of samples are underpowered but adequate to detect a trend of gene expression for further analysis (21). There was no blinding in any of the experiments.

Reads were quality checked using FastQC v0.11.5 and mapped to a human (hg19) or hybrid human-mouse (hg19-mm10) genome with STAR2.5.2b (22) using standard settings for paired reads. Uniquely mapped reads were assigned to annotated genes with HTSeq v0.6.1p1 (23) with default settings. Read counts were normalized by library size, and differential gene expression analysis based on a negative binomial distribution was performed using DESeq2 v3.4 (24). In general, thresholds for differential expression were set as follows: adjusted p-value<0.05, fold change >2.0 or <0.5, and average normalized read count >10. Genes were considered detectable in the immunoprecipitation samples with a normalized read count >100. Gene set enrichment analysis was performed using GSVA v3.4 (25) and previously curated gene sets (26): HALLMARK_TGF_BETA_SIGNALING, HALLMARK_HEDGEHOG_SIGNALING, HALLMARK_WNT_BETA_CATENIN_SIGNALING, HALLMARK_PI3_K_AKT_MTOR_SIGNALING, HALLMARK_NOTCH_SIGNALING, HALLMARK_KRAS_SIGNALING_UP, CORDENONSI_YAP_CONSERVED_SIGNATURE, HALLMARK_IL6_JAK_STAT3_SIGNALING.

### Plasmids

Cytosine Deaminase (CD)(Addgene 35102), Uracil Phospho Ribosyl Transferase (UPRT) (Addgene 47110) and rtTA3 (Addgene 26730) expressing plasmids were purchased from Addgene. Primers used for cloning the constructs described in the manuscript are described in Table S4. CD and UPRT described above were used as template for PCR for subcloning. RFP and IRES were amplified using pTRIPZ (Dharmacon) as a template. The PCR products were either ligated using DNA Ligase after restriction enzyme digestion and/or by Gibson Assembly.

### REFERENCE LIST

1. Gay, L. et al. Mouse TU tagging: a chemical/genetic intersectional method for purifying cell type-specific nascent RNA. Genes Dev. 27, 98-115 (2013).
2. Bertin, B., Renaud, Y., Aradhya, R., Jagla, K. & Junion, G. TRAP-rc, Translating Ribosome Affinity Purification from Rare Cell Populations of Drosophila Embryos. J. Vis. Exp. JoVE (2015). doi:10.3791/52985
3. Okaty, B. W., Sugino, K. & Nelson, S. B. Cell Type-Specific Transcriptomics in the Brain. J. Neurosci. 31, 6939-6943 (2011).
4. Ke, R., Mignardi, M., Hauling, T. & Nilsson, M. Fourth Generation of Next-Generation Sequencing Technologies: Promise and Consequences. Hum. Mutat. 37, 1363-1367 (2016).
5. Crosetto, N., Bienko, M. & van Oudenaarden, A. Spatially resolved transcriptomics and beyond. Nat. Rev. Genet. 16, 57-66 (2015).
6. Lee, J. H. et al. Highly Multiplexed Subcellular RNA Sequencing in Situ. Science 343, 1360-1363 (2014).
7 Mullen, C. A., Kilstrup, M. & Blaese, R. M. Transfer of the Bacterial Gene for Cytosine Deaminase to Mammalian Cells Confers Lethal Sensitivity to 5-Fluorocytosine: A Negative Selection System. Proc. Natl. Acad. Sci. U.S.A 89, 33-37 (1992).
8. Aten, J. A., Bakker, P. J., Stap, J., Boschman, G. A. & Veenhof, C. H. DNA double labelling with IdUrd and CldUrd for spatial and temporal analysis of cell proliferation and DNA replication. Histochem. J. 24, 251-259 (1992).
9. Wohlhueter, R. M., Ivor, R. S. M. & Plagemann, P. G. W. Facilitated transport of uracil and 5-fluorouracil, and permeation of orotic acid into cultured mammalian cells. J. Cell. Physiol. 104, 309-319 (1980).
10. Ojugo, A. S. et al. Influence of pH on the uptake of 5-fluorouracil into isolated tumour cells. Br. J. Cancer 77, 873-879 (1998).
11. Yuasa, H., Matsuhisa, E. & Watanabe, J. Intestinal brush border transport mechanism of 5-fluorouracil in rats. Biol. Pharm. Bull. 19, 94-99 (1996).
12. Kaehler, C., Isensee, J., Hucho, T., Lehrach, H. & Krobitsch, S. 5-Fluorouracil affects assembly of stress granules based on RNA incorporation. Nucleic Acids Res. gku264 (2014). doi:10.1093/nar/gku264
13. Longley, D. B., Harkin, D. P. & Johnston, P. G. 5-Fluorouracil: mechanisms of action and clinical strategies. Nat. Rev. Cancer 3, 330-338 (2003).
14. Rose, M. G., Farrell, M. P. & Schmitz, J. C. Thymidylate synthase: a critical target for cancer chemotherapy. Clin. Colorectal Cancer 1, 220-229 (2002).
15. Anderson, P. & Kedersha, N. Stress granules: the Tao of RNA triage. Trends Biochem. Sci. 33, 141-150 (2008).
16. Gay, L., Karfilis, K. V., Miller, M. R., Doe, C. Q. & Stankunas, K. Applying thiouracil (TU)-tagging for mouse transcriptome analysis. Nat. Protoc. 9, 410-420 (2014).
17. Andes, D. & Ogtrop, M. van. In Vivo Characterization of the Pharmacodynamics of Flucytosine in a Neutropenic Murine Disseminated Candidiasis Model. Antimicrob. Agents Chemother. 44, 938-942 (2000).
18. Miller, M. R., Robinson, K. J., Cleary, M. D. & Doe, C. Q. TU-tagging: cell type-specific RNA isolation from intact complex tissues. Nat. Methods 6, 439-441 (2009).
19. Perrone, L. A., Szretter, K. J., Katz, J. M., Mizgerd, J. P. & Tumpey, T. M. Mice Lacking Both TNF and IL-1 Receptors Exhibit Reduced Lung Inflammation and Delay in Onset of Death following Infection with a Highly Virulent H5N1 Virus. J. Infect. Dis. 202, 1161-1170 (2010).
20. Miller, M. R., Robinson, K. J., Cleary, M. D. & Doe, C. Q. TU-tagging: cell type-specific RNA isolation from intact complex tissues. Nat. Methods 6, 439-441 (2009).
21. Li, C.I., Su, P. F. & Richter, J. R. Sample size calculation based on exact test for assessing differential expression analysis in RNA-seq data. BMC Bioinformatics 14, 357 (2013).
22. Dobin, A., Davis, C.A., Schlesinger, F., Drenkow, J., Zaleski, C., Jha, S., Batut, P., Chaisson, M. & Gingeras, T. R. STAR: ultrafast universal RNA-seq aligner. Bioinforma. Oxf. Engl. 29, 15-21 (2013)
23. Anders, S. & Huber, W. Differential expression analysis for sequence count data. Genome Biol. 11. R106 (2010).
24. Love, M. I., Huber, W. & Anders, S. Moderated estimation of fold change and dispersion for RNA-seq data with DESeq2. Genome Biol. 15, 550 (2014).
25. Hanzelmann, S., Castelo, R. & Guinney, J. GSVA: gene set variation analysis for microarray and RNA-Seq data. BMC Bioinformatics 14, 7 (2013).
26. Subramanian, A. et al. Gene set enrichment analysis: A knowledge-based approach for interpreting genome-wide expression profiles. Proc. Natl. Acad. Sci. 102, 15545-15550 (2005).

1. A method of producing tagged RNA from mammalian cells of interest present in a tissue or tissue culture that contains multiple cell types, the method comprising contacting a tissue or a tissue culture that contains multiple cell types with an effective amount of a halogenated cytosine, wherein mammalian cells of interest in the tissue or tissue culture have been engineered to express a recombinant cytosine deaminase enzyme, thereby generating halogenated uridine-tagged RNA in the cells of interest.

2. The method of claim 1, further comprising subsequently separating halogenated uridine-tagged RNA from other components of the tissue, tissue culture, and/or cells of interest.

3. The method of claim 1, further comprising subsequently contacting a cell lysate or RNA sample derived from the tissue or tissue culture with an antibody that binds specifically to halogenated uridine-tagged RNA.

4. The method of claim 3, further comprising subsequently separating halogenated uridine-tagged RNA from other components in the cell lysate or RNA sample based on its binding to the antibody, thereby obtaining halogenated uridine-tagged RNA from the cells of interest present in the tissue or tissue culture.

5. A method of obtaining tagged RNA from mammalian cells of interest present in a tissue or tissue culture that contains multiple cell types, the method comprising: (a) contacting a tissue or a tissue culture that contains multiple cell types with an effective amount of a halogenated cytosine, wherein mammalian cells of interest in the tissue or tissue culture have been engineered to express a recombinant cytosine deaminase enzyme; (b) contacting a cell lysate or RNA sample derived from the tissue or tissue culture with an antibody that binds specifically to halogenated uridine-tagged RNA; and (c) separating halogenated uridine-tagged RNA from other components in the cell lysate or RNA sample based on its binding to the antibody; thereby obtaining tagged RNA from the cells of interest present in the tissue or tissue culture.

6. The method of claim 1, further comprising contacting the tissue or tissue culture with an effective amount of exogenous thymine.

7. The method of claim 1, wherein the cells of interest also express a recombinant uracil phosphoribosyltransferase (UPRT) enzyme.

8. A method of producing tagged RNA from mammalian cells of interest present in a tissue or tissue culture that contains multiple cell types, the method comprising: (a) contacting a tissue or a tissue culture that contains multiple cell types with an effective amount of a halogenated cytosine, wherein mammalian cells of interest in the tissue or tissue culture have been engineered to express a recombinant cytosine deaminase enzyme, and (b) contacting the tissue or a tissue culture with an effective amount of exogenous thymine, thereby producing tagged RNA from the cells of interest present in the tissue or tissue culture.

9. A method of producing tagged RNA from mammalian cells of interest present in a tissue or tissue culture that contains multiple cell types, the method comprising: (a) contacting a tissue or a tissue culture that contains multiple cell types with an effective amount of a halogenated cytosine, wherein mammalian cells of interest in the tissue or tissue culture have been engineered to express both (i) a recombinant cytosine deaminase enzyme, and (ii) a recombinant uracil phosphoribosyltransferase (UPRT) enzyme, and (b) contacting the tissue or a tissue culture with an effective amount of an exogenous thymine, thereby producing tagged RNA from the cells of interest present in the tissue or tissue culture.

10. The method of any one of the preceding claims, further comprising performing RNA sequencing of the tagged RNA.

11. The method of any one of the preceding claims, further comprising reverse transcribing the tagged RNA to produce cDNA.

12. The method of any one of the preceding claims, further comprising performing RT-PCR with the tagged RNA.

13. The method of any one of the preceding claims, further comprising amplifying the tagged RNA, or cDNA derived therefrom.

14. The method of any one of the preceding claims, further comprising performing microarray analysis of the tagged RNA.

15. The method of claim 1, wherein the halogenated cytosine is selected from the group consisting of fluoro-cytosine, chloro-cytosine, bromo-cytosine, and iodo-cytosine.

16. The method of claim 1, wherein the halogenated cytosine is selected from the group consisting of 5-fluoro-cytosine, 5-chloro-cytosine, 5-bromo-cytosine, and 5-iodo-cytosine.

17. The method of claim 1, wherein the halogenated cytosine is 5-fluoro-cytosine and wherein the halogenated uridine-tagged RNA is 5-fluoro-uridine-tagged RNA.

18. The method of claim 1, further comprising contacting a cell lysate or RNA sample derived from the tissue or tissue culture with an anti-BrdU antibody.

19. The method of claim 1, further comprising performing immuno-affinity chromatography to separate the halogenated uridine-tagged RNA from other components in the cell lysate or RNA sample.

20. The method of claim 19, comprising performing two successive rounds of immuno-affinity chromatography to separate the halogenated uridine-tagged RNA from other components in the cell lysate or RNA sample.

21. The method of claim 1, comprising performing immunoprecipitation to separate the halogenated uridine-tagged RNA from other components in the cell lysate or RNA sample.

22. The method of claim 22, comprising performing two successive rounds of immunoprecipitation to separate the halogenated uridine-tagged RNA from other components in the cell lysate or RNA sample.

23. The method of claim 1, wherein the cells of interest comprise a recombinant nucleic acid molecule that comprises a nucleotide sequence encoding a cytosine deaminase enzyme operatively linked to a promoter.

24. The method of claim 1, wherein the cells of interest comprise a recombinant nucleic acid molecule that comprises a nucleotide sequence encoding a UPRT enzyme operatively linked to a promoter.

25. The method of claim 1, wherein the cells of interest comprise both (a) a recombinant nucleotide sequence encoding a cytosine deaminase enzyme, and (b) a recombinant nucleotide sequence encoding a UPRT enzyme.

26. The method of claim 25, wherein the nucleotide sequence encoding the cytosine deaminase enzyme and the nucleotide sequence encoding the UPRT enzyme are present on the same nucleic acid molecule.

27. The method of claim 26, wherein the nucleic acid molecule comprises an internal ribosome entry site (IRES) sequence or viral 2A peptide encoding sequence located between the nucleotide sequence encoding the cytosine deaminase enzyme and the nucleotide sequence encoding the UPRT enzyme.

28. The method of claim 25, wherein the nucleotide sequence encoding the cytosine deaminase enzyme and the nucleotide sequence encoding the UPRT enzyme are each present on a separate nucleic acid molecule.

29. The method of any of claims 23-28 wherein the nucleic acid molecule comprises an inducible promoter.

30. The method of any of claims 23-28 wherein the nucleic acid molecule comprises a tissue-specific promoter.

31. The method of claim 1, wherein the cells of interest are in vivo.

32. The method of claim 31, wherein the tissue is contacted with the halogenated cytosine in vivo.

33. The method of any claim 1, wherein the cells of interest have been injected into a living animal, or are derived from cells that have been injected into a living animal.

34. The method of claim 1, wherein the cells of interest are present in a genetically engineered animal that has been engineered to express the recombinant cytosine deaminase enzyme.

35. The method of claim 1, wherein the cells of interest are present in a genetically engineered animal that has been engineered to express the recombinant cytosine deaminase enzyme and UPRT.

36. The method of claim 1, wherein the cells of interest are in vitro.

37. The method of claim 36, wherein the tissue or tissue culture is contacted with the halogenated cytosine in vitro.

38. The method of claim 37, wherein the tissue or tissue culture is contacted in vitro with the halogenated cytosine at a concentration of up to about 50 micro molar.

39. The method of claim 37, wherein the tissue or tissue culture is contacted in vitro with the halogenated cytosine at a concentration of up to about 250 micro molar.

40. The method of claim 37, wherein the tissue or tissue culture is contacted in vitro with the halogenated cytosine at a concentration of up to about 500 micro molar.

41. The method of claim 32, wherein the tissue is contacted in vivo with the halogenated cytosine at a concentration of up to about 50 mg/kg.

42. The method of claim 32, wherein the tissue or tissue culture is contacted in vivo with the halogenated cytosine at a concentration of up to about 250 mg/kg.

43. The method of claim 32, wherein the tissue or tissue culture is contacted in vivo with the halogenated cytosine at a concentration of up to about 500 mg/kg.

44. The method of claim 32 or claim 37, wherein the tissue or tissue culture is contacted with the halogenated cytosine in vitro or in vivo for a period of at least about 2 hours.

45. The method of claim 32 or claim 37, wherein the tissue or tissue culture is contacted with the halogenated cytosine in vitro or in vivo for a period of up to about 48 hours.

46. The method of claim 32 or claim 37, wherein the tissue or tissue culture is contacted with the halogenated cytosine in vitro or in vivo for a period of up to about 24 hours.

47. The method of claim 32 or claim 37, wherein the tissue or tissue culture is contacted with the halogenated cytosine in vitro or in vivo for a period of up to about 12 hours.

48. The method of claim 32 or claim 37, wherein the tissue or tissue culture is contacted with the halogenated cytosine in vitro or in vivo for a period of up to about 8 hours.

49. The method of claim 32 or claim 37, wherein the tissue or tissue culture is contacted with the halogenated cytosine in vitro or in vivo for a period of up to about 6 hours.

50. The method of claim 32 or claim 37, wherein the tissue or tissue culture is contacted with the halogenated cytosine in vitro or in vivo for a period of up to about 4 hours.

51. The method of claim 32 or claim 37, wherein the tissue or tissue culture is contacted with the halogenated cytosine in vitro or in vivo for a period of from about 2 hours to about 24 hours.

52. The method of claim 32 or claim 37, wherein the tissue or tissue culture is contacted with the halogenated cytosine in vitro or in vivo for a period of from about 2 hours to about 12 hours.

53. The method of claim 32 or claim 37, wherein the tissue or tissue culture is contacted with the halogenated cytosine in vitro or in vivo for a period of from about 2 hours to about 8 hours.

54. The method of claim 32 or claim 37, wherein the tissue or tissue culture is contacted with the halogenated cytosine in vitro or in vivo for a period of from about 2 hours to about 6 hours.

55. The method of claim 32 or claim 37, wherein the tissue or tissue culture is contacted with the halogenated cytosine in vitro or in vivo for a period of from about 2 hours to about 4 hours.

56. The method of claim 37, wherein the tissue or tissue culture is contacted in vitro with exogenous thymine at a concentration of up to about 125 micro molar.

57. The method of claim 32, wherein the tissue is contacted in vivo with exogenous thymine at a concentration of up to about 125 mg/kg.

58. The method of claim 6, wherein the tissue or tissue culture is contacted with the halogenated cytosine and with the thymine concurrently.

59. A kit for obtaining tagged RNA from mammalian cells of interest present in a tissue or tissue culture that contains multiple mammalian cell types, the kit comprising two or more components selected from the group consisting of (a) a halogenated cytosine, (b) thymine, (c) a nucleotide molecule encoding a cytosine deaminase enzyme, (d) a nucleotide molecule encoding a UPRT enzyme, and (e) an antibody that binds to halogenated uridine-tagged RNA.

60. The kit of claim 59, wherein the halogenated cytosine is 5-fluoro-cytosine.

61. The kit of claim 59, wherein the antibody is an anti-BrdU antibody.

62. The kit of claim 59, further comprising instructions for tagging RNA in, and/or obtaining RNA from, mammalian cells of interest present in a tissue or tissue culture that contains multiple mammalian cell types.

63. A substantially pure sample of halogenated uridine-tagged RNA.

64. A substantially pure sample of 5-fluoro-uridine-tagged RNA.
